(19) United States
(12) Patent Application Publication
(10) Pub. No.: US 2018/0214605 A1
(43) Pub. Date: Aug. 2, 2018

(54) TISSUE ENDOPROSTHESIS AND METHOD FOR THE PRODUCTION THEREOF

(71) Applicant: Carmat, Vélizy Villacoublay cedex (FR)

(72) Inventors: Antoine Capel, Dourdan (FR); Clément Ducros, Viroflay (FR); Philippe Pouletty, Paris (FR); Jean-Claude Cadudal, Clamart (FR)

(73) Assignee: Carmat, Vélizy Villacoublay cedex (FR)

(21) Appl. No.: 15/544,386

(22) PCT Filed: Mar. 7, 2016
(86) PCT No.: PCT/FR2016/050525

§ 371 Date: Jul. 18, 2017

(30) Foreign Application Priority Data
Mar. 10, 2015 (FR) 1500457

(51) Int. Cl. A61L 27/36; A61F 2/24; A61F 2/90; A61F 2/07; A61L 31/14; A61L 31/10; A61L 31/00; A61L 27/50; A61L 27/34

(52) U.S. Cl. CPC A61L 27/3625; A61L 2420/02; A61F 2/90; A61F 2/07; A61L 31/14; A61L 31/10; A61L 31/005; A61L 27/50; A61L 27/34; A61F 2002/072; A61F 2210/0076; A61F 2250/0036; A61F 2240/001; A61F 2210/0057; A61L 2400/18; A61F 2/24

(57) ABSTRACT

According to the invention, said tissue endoprosthesis comprises an expandable support structure or stent held between an inner tissue structure and an outer coating consisting of a hemocompatible soft synthetic material which impregnates said inner structure through the openings in said support structure.

The present invention relates to a tissue endoprosthesis and to a method for the production of this endoprosthesis.

The tissue endoprostheses to which the present invention relates may be, for example, vascular endoprostheses or cardiac valve, especially aortic, mitral or tricuspid valve, prostheses.

There is already known from document WO 03/007781 (PCT/US02/20037) a vascular tissue endoprosthesis which comprises an expandable hollow support structure having a surface with openings, and an inner tissue structure of biological tissue which covers the inner surface of said support structure.

Said expandable hollow support structure, which is generally called a stent, has shape memory in order that it can be implanted in a vessel in a folded state and assume its radially expanded shape once it is in place in said vessel, and its surface is provided with openings due to the fact that the surface is generally composed of a mesh lattice of a material such as Nitinol, titanium, etc. The inner tissue structure, for its part, is composed, for example, of a membrane of animal peritoneum, pleura or pericardium, which is rolled up in order to be able to adapt to the shape of the inner surface of the support structure when the support structure has its radially expanded form. As a result, the inner structure of biological tissue necessarily has at least one joining line along which two opposite edges of said membrane are connected.

In this known vascular endoprosthesis, the two opposite edges so connected are secured to one another by stitches, which on the one hand are liable to cause turbulence in the flow of blood in the endoprosthesis, and therefore the formation of clots, and on the other hand cannot ensure strict tightness along said joining line. In an attempt to remedy these disadvantages, it is proposed in this earlier document to cover the line of stitches with a layer of biological glue. However, such a biological glue is a foreign product for the endoprosthesis, providing purely chemical adhesion which denatures the biological tissue and which is liable to disintegrate. This has the result that particles of biological glue which have become detached from the layer of biological glue covering the line of stitches may form blood clots, leading to an embolism for the patient.

In addition, in this known endoprosthesis, the inner tissue structure is preferably also fixed to said support structure by means of stitches. It goes without saying that these additional stitches may likewise cause blood clots, as explained above for the stitches of the inner structure. Therefore, it is additionally proposed in that document to glue said inner tissue structure to the support structure by means of a biological glue. However, the above-mentioned risks of embolism are then again found due to the use of such a biological glue.

It will be noted that the operation of producing the stitches is, moreover, intricate and lengthy, so that the production of these endoprostheses is not very efficient, with many rejects.

Furthermore, once the support structure of an endoprosthesis has been placed in the expanded position in a blood vessel, it is in contact with said vessel, pressing it radially outwards. This results in friction, which is liable to erode the surface of said vessel and may lead to displacement, inside said vessel, of the vascular endoprosthesis in the expanded state.

In addition, there is known from documents U.S. Pat. No. 5,411,552 and EP 0 850 607 A1 a cardiac valve prosthesis comprising an expandable outer support structure having a surface with openings, and an inner tissue structure at least partly covering the inner surface of said support structure. In these known valve prostheses, similarly to that described above, the inner tissue structure is fixed to the outer support structure by gluing or by producing stitches. They therefore have all the disadvantages mentioned above.

The object of the present invention is to remedy all those disadvantages by describing a perfectly tight, reinforced tissue endoprosthesis which is not liable to erode the cells of the tissues of a patient after implantation and which does not include either stitches or biological glue.

To this end, according to the invention, the tissue endoprosthesis comprising:
an expandable hollow support structure having a surface with openings (stent); and
an inner tissue structure at least partly covering the inner surface of said support structure
   is remarkable in that it comprises an outer covering which is composed of a flexible haemocompatible synthetic material and is anchored mechanically to said inner tissue structure, said support structure being held at least partially between said outer covering and said inner tissue structure.

Preferably, such mechanical anchoring is due to the fact that the flexible haemocompatible synthetic material of said outer covering impregnates said inner tissue structure at least partially.

Accordingly, in the tissue endoprosthesis according to the present invention, said outer covering, which is made of a pure or loaded (inclusion, active substance, etc.), flexible synthetic material, advantageously an elastomer, preferably a polyurethane elastomer or a silicone elastomer, performs several functions without, by virtue of its flexibility, impeding the expansion of said expandable support structure (stent) from its folded implantation position to its expanded implanted position. In fact, said outer covering:
which has access to said inner tissue structure through the openings in the surface of the support structure, produces with the inner structure bonding by mechanical anchoring due to the impregnation of the inner structure by the flexible synthetic material of said outer covering, said bonding, which holds said support structure and secures it to the outer covering and to the inner structure, making it possible to obtain very good mechanical adhesion, in contrast to a biological glue, which causes purely chemical adhesion;
covers the joining line, which is the result of the shaping of the inner covering from a membrane, by adhering to the edges facing the joining line and thus ensures the tightness of the joining line; and
forms, for the endoprosthesis according to the present invention, a flexible outer reinforcement which, after implantation, protects the natural walls of the patient from wear resulting from friction.

Advantageously, the thickness of said outer covering is at least 0.1 mm and may reach 5 mm. Optionally, the thickness may be non-uniform and vary from one location to another of said outer covering.

The inner tissue structure can be composed of a biological tissue of animal origin, for example pericardium, or of a synthetic material, for example polytetrafluoroethylene.

According to the present invention, said tissue endoprosthesis is advantageously produced by carrying out a method which is remarkable in that:
said inner tissue structure is introduced into said support structure so as to cover in a continuous, uniform manner, without folding or excessive thickness, at least a portion of the inner surface of said support structure;
by means of a dispersion of a flexible haemocompatible synthetic material in a solvent, the surface of said support structure and the portions of the outer surface of said inner tissue structure that are accessible through the openings in the surface of said support structure are covered in order to form an outer covering of said flexible haemocompatible synthetic material which is anchored mechanically in said inner tissue structure; and
said solvent is extracted from said outer covering.

In the case where said inner tissue structure is formed by a chemically fixed biological tissue, the inner tissue structure is subjected to partial dehydration after chemical fixing and before introduction into said support structure and is then rehydrated during or after the extraction of the solvent from said outer covering.

Advantageously, the concentration by weight of said synthetic material in the dispersion is between 10 and 30%, preferably between 20 and 22%. The viscosity of said dispersion is advantageously between 500 and 1000 cP.

The figures of the accompanying drawing explain how the invention can be carried out. In the figures, identical reference numerals denote similar elements.

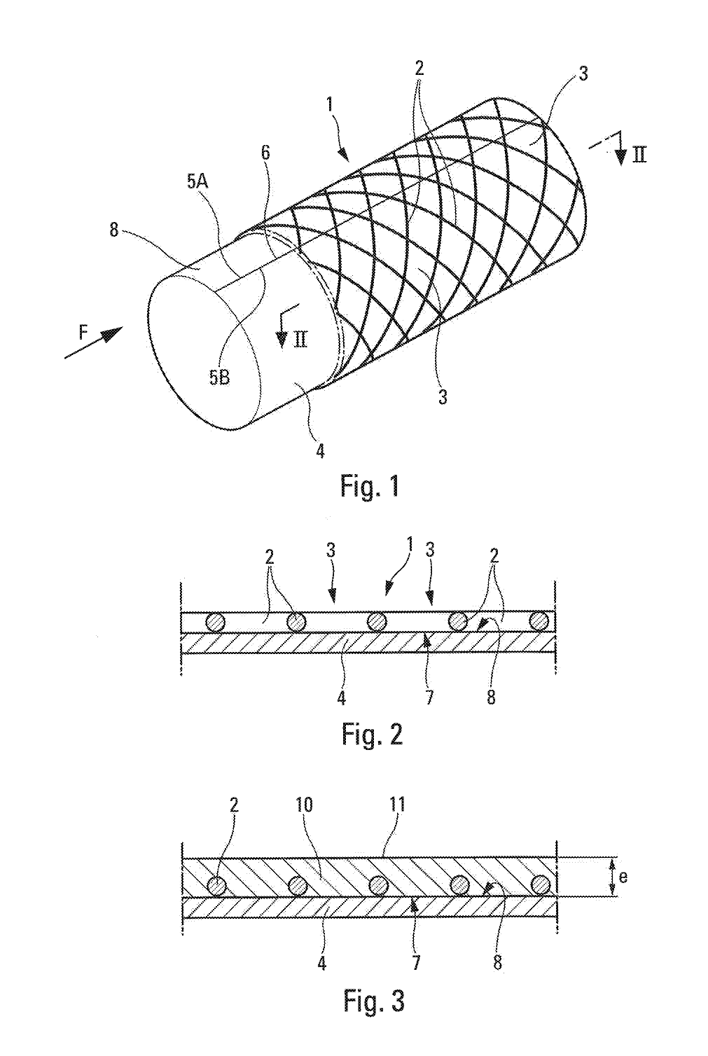

FIG. 1 shows schematically and in part a portion of an endoprosthesis during fitting of the inner tissue structure into the expandable support structure.

FIG. 2 is a partial longitudinal section, along line II-II of FIG. 1, of the wall of the support structure/inner tissue structure assembly after assembly of those elements.

FIG. 3 corresponds to FIG. 2 and shows the outer covering produced on said support structure/inner tissue structure assembly, according to the present invention.

FIG. 1 shows, in the deployed position, a portion of a tissue endoprosthesis, for example a vascular endoprosthesis or a cardiac valve endoprosthesis, comprising:
a support structure 1 (stent) composed of a lattice of wires 2 of an alloy with shape memory (steel, Nitinol, metal, polymer, etc.), the meshes 3 of which form openings in the surface of said support structure, it being possible for the structure to be symmetrical or asymmetrical in the longitudinal or lateral axis; and
an inner tissue structure 4, for example cut from a membrane (not shown) of biological tissue and rolled so that its opposite edges 5A and 5B meet along a joining line 6, it being possible for the biological tissue to be of animal origin (pig, horse, cattle, etc.) or produced by tissue culture from human cells. Good results have been obtained with animal pericardium. However, the inner tissue structure 4 may be composed of a synthetic material, such as, for example, expanded polytetrafluoroethylene.

In FIG. 1 there has been shown, schematically, the axial introduction, according to arrow F, of the inner structure 4, in the deployed position, into the expandable support structure (stent) 1, which is itself in the expanded position.

When, as is shown schematically in FIG. 2, the inner structure 4 is in place in the support structure 1, it covers at least partly the inner surface 7 of said support structure 1, that is to say the surface formed by said wires 2, the outer surface 8 of said inner structure 4 remaining accessible, however, from the outside through the openings formed by the meshes 3 of said support structure 1.

The production of the tissue endoprosthesis according to the present invention comprises a plurality of steps:

1. Said inner structure 4 is first shaped to the expanded form which it must have when the support structure 1 is itself expanded (FIG. 1), for example by winding on a mandrel;

2. In the case where said inner tissue structure is formed by a biological tissue, preferably animal pericardium, the biological tissue is fixed chemically, in a known manner, by any suitable product such as an aldehyde. In the latter case, glutaraldehyde is preferably used, for example in a concentration of 0.625%. Such chemical fixing ensures that the biological tissue has reduced antigenicity, chemical, biological and physical stability, and especially resistance to fluctuations in temperature and mechanical stresses;

3A. The biological tissue of said inner structure 4 is then lyophilised. The aim of this treatment is on the one hand to dehydrate the tissue, which is essential in order for it to adhere, but also to preserve the three-dimensional structure of said biological tissue after dehydration. When a biological tissue dehydrates under ordinary conditions, the collagen fibres of which it is composed come into contact with one another and irreversible chemical bonds are formed, making subsequent rehydration of the biological tissue impossible. In order to avoid this disadvantage, lyophilisation makes it possible to immobilise the structure of the biological tissue by freezing and then to remove the water at very low pressure by sublimation, therefore without permitting mobility and thus rearrangement of the fibres. The two parameters which are essential for controlling the lyophilisation are kinetics and dehydration:
the kinetics must be very slow—for example between 2° C./hour and 8° C./hour—in order to avoid any risk of supercooling (melting of the water leading to local rehydration);
the rate of dehydration must be at least 75%, preferably approximately 78 to 80%. A rate of dehydration which is too low would permit a certain degree of rehydration of the biological tissue and therefore a certain degree of mobility of the fibres, which would result in a loss of flexibility and poor rehydration after a storage phase (even of short duration). Excessive dehydration, in turn, would denature the biological material and lead to considerable shrinkage. Biological materials are in fact composed of bound water (10% dry matter and 10% bound water in the case of the pericardium), which is part of the very composition of the material and must therefore not be removed.

In order to improve the conservation of the tissue structure even further during lyophilisation, the biological tissue is first treated for several days with a glycol, advantageously polyethylene glycol, before being lyophilised. Polyethylene glycol will create low-energy bonds with the various collagen fibres and therefore interpose itself between the fibres like the rungs of a ladder. During lyophilisation, the various fibres are therefore unable to interact with one another. However, because the bonds are low-energy bonds, polyethylene glycol, although perfectly biocompatible (for some molecular masses, according to the European pharmacopoeia), is easily rinsed off during rehydration;

3B. In a variant, the lyophilisation can be replaced by slow chemical dehydration by immersion of said inner covering 4 in an alcoholic solution of polyethylene glycol having a concentration of at least 80% polyethylene glycol and 10% alcohol, for example. Dehydration is then carried out at low pressure and at a stable temperature, for example 40° C., for a minimum of 12 hours.

4. Thus, by virtue of the dehydration of step 3A or that of step 3B, there is obtained an inner tissue structure 4 of dry biological tissue which is perfectly rehydratable without alteration of said biological tissue and virtually without surface shrinkage. As illustrated schematically by FIG. 1, this inner tissue structure 4 is then introduced into the support structure 1, for example by means of said mandrel (not shown) on which it is wound. It is then flattened (by any known means such as an inflatable balloon, expandable mandrel, etc. not shown) against said support structure 1 so as to form a continuous, uniform covering, without folding or excessive thickness, with the edges 5A and 5B touching one another exactly to form a continuous and even joining line 6 without overlapping or gapping. As is shown in FIG. 2, in this situation the outer surface 8 of the inner tissue structure 4 in the deployed position is then applied perfectly to the inner surface 7 of the support structure 1, likewise in the expanded position, the outer surface 8 nevertheless remaining accessible from the outside through the meshes 3.

5. There is then formed on the support structure 1/inner tissue structure 4 assembly obtained in step 4 above an outer covering 10 which holds the support structure 1, securing the latter to the inner tissue structure 4, owing to the covering of the outer surface 8 of the inner tissue structure 4 through the meshes 3 of said support structure 1 and, in addition, sealing the joining line 6. This outer covering 10 is formed by deposition of an adhesion agent formed by a dispersion of a flexible and biocompatible synthetic material in a solvent dependent thereon. This flexible synthetic material may be an implantable biocompatible polyurethane elastomer and the solvent may then be dimethylacetamide.

In a variant, the flexible synthetic material of said dispersion may be a silicone elastomer, for example implantable biocompatible polydimethylsiloxane, and the solvent may then be xylene.

The concentration by weight of synthetic material in said dispersion is advantageously between 10 and 30%, preferably between 20 and 22%. The viscosity of the dispersion is adjusted between 500 and 1000 cP according to the nature of the biological tissue of the inner structure 4 and according to the mode of deposition.

The outer covering 10 can be formed, starting from said dispersion, by any known means, for example coating, dipping, pouring or atomisation, according to the desired surface condition and the viscosity of the dispersion.

Preferably, the outer covering 10 is produced by superposing a plurality of consecutive layers until the desired thickness e is obtained, which is, for example, between 0.1 and 5 mm.

The outer surface 11 of the outer covering 10 is continuous and preferably has a microporosity capable of inducing slight fibrosis in order to strengthen the mechanical bond with the natural wall of the patient in whom the endoprosthesis is implanted. Such external microporosity can be obtained by atomising a low-viscosity dispersion having water-soluble inclusions. The particle size of the inclusions must be controlled in order to control the size of the pores of the microporosity.

The outer surface 11 of the outer covering 10 may optionally comprise one or more outer embossment(s) capable of improving the mechanical holding of the implanted endoprosthesis. However, in this case it must be ensured that the embossment(s) does/do not induce cell erosion of the natural wall of the patient.

6. After the outer covering 10 has been formed, the solvent is removed from said dispersion, for example by drying at elevated temperature, drying at elevated temperature in vacuo and/or by extraction at elevated temperature in physiological serum. Preferably, the solvent is removed by slow extraction at elevated temperature (for example at a temperature of approximately 40° C.), followed by extraction in vacuo and completed by extraction in physiological serum.

Finally, during or after the phase of extraction of the solvent, the inner covering 4 is rehydrated with physiological serum.

The outer face 11 of the outer covering 10 may optionally be grafted chemically with peptides, proteins or molecules promoting cell adhesion and serum proteins. This outer face may additionally be coated with a wetting agent or with a biocompatible lubricant which facilitates sliding of the endoprosthesis during loading of the implantation catheter or displacement for positioning of the endoprosthesis.

Although not shown in the drawings, at least one marker locatable by medical imaging may be provided on the endoprosthesis according to the present invention in order to facilitate implantation thereof.

As mentioned above, the endoprosthesis according to the present invention may constitute all types of prosthesis, including cardiac valve, mainly aortic, mitral and tricuspid valve, prostheses.

A disadvantage of known transcatheter valves is possible post-implantation paravalvular leakage, which is detrimental to the medical prognosis. For aortic valves especially, owing to often non-homogeneous calcifications of the aortic ring, spaces may remain between the valve and the ring. In order to remedy this disadvantage, the outer covering 10 of a valve according to the invention may have optimised thickness and flexibility in order to adapt spontaneously to the contours of the native tissue or of peripheral calcifications.

In addition, a different thickness over the circumference of the valve according to the invention, or in the longitudinal axis thereof, may allow the valve prosthesis to be better adjusted to the anatomy of each patient or of sub-groups of patients.

The thickness e and the composition of the outer covering 10 of the valve prosthesis according to the present invention may be non-uniform and vary at different locations of said valve prosthesis.

1. A tissue endoprosthesis comprising:
an expandable hollow support structure having a surface with openings; and
an inner tissue structure at least partly covering the inner surface of said support structure,
wherein the tissue endoprosthesis comprises an outer covering which is composed of a flexible haemocompatible synthetic material and is anchored mechanically to said inner tissue structure, said support structure being held at least partially between said outer covering and said inner tissue structure.

2. The tissue endoprosthesis according to claim 1,
wherein the flexible haemocompatible synthetic material of said outer covering impregnates said inner tissue structure at least partially.

3. The tissue endoprosthesis according to claim 1,
wherein the flexible haemocompatible synthetic material of said outer covering is an elastomer.

4. The tissue endoprosthesis according to claim 3,
wherein said elastomer is a polyurethane elastomer or a silicone elastomer.

5. The tissue endoprosthesis according to claim 1,
wherein the flexible haemocompatible synthetic material of said outer covering is pure or loaded.

6. The tissue endoprosthesis according to claim 1,
wherein the thickness of said outer covering is at least 0.1 mm and at most 5 mm.

7. The tissue endoprosthesis according to claim 1,
wherein the thickness of said outer covering is different at different locations of said endoprosthesis.

8. The tissue endoprosthesis according to claim 1,
wherein the inner tissue structure is composed of animal pericardium.

9. The tissue endoprosthesis according to claim 1,
wherein the inner tissue structure is composed of expanded polytetrafluoro-ethylene.

10. The tissue endoprosthesis according to claim 1,
wherein the tissue endoprosthesis forms a vascular prosthesis.

11. The tissue endoprosthesis according to claim 1,
wherein the tissue endoprosthesis forms a cardiac valve prosthesis.

12. A method for the production of a tissue endoprosthesis comprising:
an expandable hollow support structure having a surface with openings; and
an inner tissue structure at least partly covering the inner surface of said support structure,
the method comprising:
introducing said inner tissue structure into said support structure so as to cover, in a continuous, uniform manner, without folding or excessive thickness, at least a portion of the inner surface of said support structure;
covering the surface of said support structure and the portions of the outer surface of said inner tissue structure that are accessible through the openings in the surface of said support structure by means of a dispersion of a flexible haemocompatible synthetic material in a solvent in order to form an outer covering of said flexible haemocompatible synthetic material which is anchored mechanically in said inner covering; and
extracting said solvent from said outer covering.

13. The method according to claim 12,
wherein said synthetic material of the outer covering is a polyurethane elastomer or a silicone elastomer.

14. The method according to claim 12,
wherein the concentration by weight of said synthetic material in the dispersion is between 10 and 30%.

15. The method according to claim 14,
wherein the concentration by weight of said synthetic material in the dispersion is between 20 and 22%.

16. The method according to claim 12,
wherein the viscosity of said dispersion is between 500 and 1000 cP.

17. The method according to claim 12, wherein said inner tissue structure is formed by a chemically fixed biological tissue,
wherein said inner tissue structure:
is subjected to partial dehydration after chemical fixing and before introduction into said support structure; and then
is rehydrated during or after the extraction of the solvent from said outer covering.
